(19) United States
(12) Patent Application Publication
(10) Pub. No.: US 2020/0246493 A1
(43) Pub. Date: Aug. 6, 2020

(54) DRUG-IODINATED OIL DISPERSION, METHOD FOR PREPARING SAME AND ITS APPLICATION IN EMBOLIZATION TREATMENT FOR LIVER CANCER

(71) Applicant: Xiamen University, Xiamen (CN)

(72) Inventors: Gang LIU, Xiamen (CN); Chengchao CHU, Xiamen (CN); Jing ZHU, Xiamen (CN); Yang ZHANG, Xiamen (CN); Aizheng CHEN, Xiamen (CN); Biaoqi CHEN, Xiamen (CN)

(21) Appl. No.: 16/581,600

(22) Filed: Sep. 24, 2019

(30) Foreign Application Priority Data
Feb. 1, 2019 (CN) 201910105683.X

(51) Int. Cl. A61K 51/12

(52) U.S. Cl. CPC A61K 51/1217; A61K 9/0019

(57) ABSTRACT

Disclosed is a method of preparing a drug-iodinated oil dispersion by high-pressure dissolution. In the invention, drug molecules are dissolved in and thoroughly mixed with iodinated oil under high pressure and rapid stirring. After the depressurization, the drug molecules are dispersed in the iodinated oil to prepare a uniformly-mixed drug-iodinated oil dispersion. The drug-iodinated oil dispersion prepared herein, compared to the drug-iodinated oil suspension and emulsion, has advantages of controllable morphology, long-term stability and slow drug-releasing rate, moreover, the drug molecules do not negatively affect the iodinated oil in physicochemical properties such as viscosity. In the liver cancer treatment, the prepared drug-iodinated oil dispersion is firstly injected through the hepatic arteries to perform the embolization, and the drug molecules are slowly released for a sustained treatment. The invention provides a good reference for the therapy combined with iodinated oil embolization.

### CROSS-REFERENCE TO RELATED APPLICATIONS

This application claims the benefit of priority from Chinese Patent Application No. 201910105683.X, filed on Feb. 1, 2019. The content of the aforementioned application, including any intervening amendments thereto, is incorporated herein by reference in its entirety.

### TECHNICAL FIELD

The application relates to the field of medicine, and more particularly to a drug-iodinated oil dispersion, a method of preparing the same by high-pressure dissolution and its application in embolization co-treatment for liver cancer.

### BACKGROUND OF THE INVENTION

Liver cancer, with a high mortality rate, occurs often at Asia and Africa areas. Surgery is generally preferred for the treatment of liver cancer. However, liver cancer is often accompanied with liver cirrhosis, which limits the precise surgical resection. In addition, it fails to treat the liver cancer in the mid-late stage by surgery. Transarterial chemoembolization (TACE) is well recognized as an important therapy for the liver cancer. Currently, due to the convenient injection and selective deposition, iodinated oil has been the most commonly-used embolic agent for TACE. Liver cancer tissues are mainly supplied with blood through hepatic arteries, while the normal liver tissues are supplied with blood through the portal vein and the hepatic arteries. In the TACE treatment, the iodinated oil deposits in the hepatic sinus, interstitial space and even small blood vessels of liver cancer tissues to block the blood supply to the tumor tissues, thereby killing the tumor cells. Moreover, the iodinated oil will be removed in time in the normal liver tissues, avoiding the occurrence of obvious damage.

Furthermore, to improve the effect of TACE treatment, the iodinated oil also could combine with chemotherapeutic drug molecules, radiotherapeutic drug molecules or probe molecules, facilitating the combination of tumor embolization with chemotherapy, radiotherapy or surgical navigation guided by imaging to provide the most effective treatment. Currently, since the drug molecules are less compatible with the iodinated oil, mechanical mixing or a co-solvent is often introduced to prepare a drug molecule-iodinated oil suspension, or an emulsifier is used to prepare a drug molecule-iodinated oil emulsion. It has been demonstrated by lots of clinical studies that the combination of the iodinated oil embolization with chemotherapy or radiotherapy can achieve effective treatment.

However, for the drug-iodinated oil suspension or emulsion, the drug molecules are both dispersed in the form of micro particle in the iodinated oil, so that the precipitation or stratification will easily occur during and after the injection of the mixture, unsuitable for the long-term treatment. In the TACE, the iodinated oil can be fully dispersed in the liver cancer area due to high viscosity, while the introduced solvent or emulsifier may reduce the viscosity of the iodinated oil, adversely affecting the treatment. Therefore, there is an urgent need in the combination of iodinated oil embolization with other treatments to develop a drug-iodinated oil dispersion involving stable dispersion, long-term deposition and slow release of drug molecules and the maintenance of high viscosity of the iodinated oil. The invention discloses a method of preparing a drug-iodinated oil dispersion by pressure dissolution, i.e., Superstable Homogeneous Iodinated Formulation Technology (SHIFT). The drug-iodinated oil dispersion prepared by SHIFT has a controllable morphology, long-term stability and a slow drug-releasing rate. Notably, the drug molecules do not obviously affect the viscosity of the iodinated oil.

### SUMMARY OF THE INVENTION

An object of the invention is to provide a drug-iodinated oil dispersion, comprising a drug molecule and an iodinated oil, where the drug molecule has a concentration of 0.05-5 mg/mL in the drug-iodinated oil dispersion; the drug-iodinated oil dispersion is prepared through the following steps:

1) dispersing the drug molecule in a first solvent to obtain a solution A;

2) adding the iodinated oil injection and the solution A to a high-pressure reactor;

3) boosting a pressure of the high-pressure reactor to 10-20 Mpa and controlling a temperature of the high-pressure reactor at 30-50° C.; and stirring the reaction mixture for 0.5-2 h after the pressure and the temperature are stable; and

4) depressurizing the high-pressure reactor; and collecting the drug-iodinated oil dispersion from the high-pressure reactor.

In some embodiments, the first solvent is ethanol.

In some embodiments, the drug molecule has a concentration of 0.5-10 mg/mL in the solution A, and a volume ratio of the solution A to the iodinated oil injection is 0.01-0.5:1-10.

In some embodiments, the iodinated oil injection comprises 37.0%-41.0% by weight of iodine.

In some embodiments, the drug molecule is selected from a chemotherapeutic drug molecule, a radiotherapeutic drug molecule, a radiotherapy-sensitized chemotherapeutic drug molecule, and a photosensitive drug molecule, or a combination thereof.

In some embodiments, the chemotherapeutic drug molecule comprises at least one of epirubicin, 5-fluorouridine, pirarubicin, pingyangmycin, gemcitabine, camptothecin and paclitaxel.

In some embodiments, the radiotherapeutic drug molecule comprises at least one of ¹³¹I-, ¹⁷⁷Lu- and ⁶⁴Cu-labeled radiotherapeutic molecules.

In some embodiments, the radiotherapy-sensitized chemotherapeutic drug molecule comprises at least one of docetaxel, curcumin, tirapazamine, cisplatin, doxorubicin and mitomycin C.

In some embodiments, the photosensitive drug molecule comprises at least one of indocyanine green, new indocyanine green IR-820, 5-aminolevulinic acid and porphyrin dye.

A second object of the invention is to provide a use method of the drug-iodinated oil dispersion, comprising:

applying the drug-iodinated oil dispersion in the preparation of a tumor-treating drug or an embolic agent.

A third object of the invention is to provide a method of preparing a drug-iodinated oil dispersion, comprising:

1) dispersing a drug molecule in ethanol to obtain a solution A;

2) adding the iodinated oil injection and the solution A to a high-pressure reactor;

3) boosting a pressure of the high-pressure reactor to 10-20 Mpa and controlling a temperature of the high-pressure reactor at 30-50° C.; and stirring the reaction mixture for 0.5-2 h after the pressure and the temperature are stable; and

4) depressurizing the high-pressure reactor; and collecting the drug-iodinated oil dispersion from the high-pressure reactor.

In some embodiments, the drug molecule has a concentration of 0.5-10 mg/mL in the solution A, and a volume ratio of the solution A to the iodinated oil injection is 0.1-0.2:2-5.

In some embodiments, the reaction mixture in step 3) is stirred at a rate of 8,000-12,000 rpm.

The invention provides a method of preparing a drug-iodinated oil dispersion in which a drug molecule is mixed with the iodinated oil to form a uniform, singly-dispersive, particle-free and stable dispersion.

In one embodiment, a chemotherapeutic drug molecule is used in the method to prepare a chemotherapeutic drug molecule-iodinated oil dispersion, which leads to an embolization in the hepatic feeding artery, so that the chemotherapeutic drug molecule-iodinated oil dispersion can be largely held in the liver cancer area for a long time to slowly release the chemotherapeutic drug molecules, thereby realizing a long-term co-treatment (more than three weeks) of embolization and chemotherapy.

In another embodiment, a radiotherapeutic drug molecule is used in the method to prepare a radiotherapeutic drug molecule-iodinated oil dispersion, which leads to an embolization in the hepatic feeding artery, so that the radiotherapeutic drug molecule-iodinated oil dispersion can be largely held in the liver cancer area for a long time to slowly release the radiotherapeutic drug molecules, thereby realizing a long-term co-treatment (more than three weeks) of embolization and radiotherapy.

In an embodiment, a radiotherapy-sensitized chemotherapeutic drug molecule is used in the method to prepare a radiotherapy-sensitized chemotherapeutic drug molecule-iodinated oil dispersion, which leads to an embolization in the hepatic feeding artery, so that the radiotherapy-sensitized chemotherapeutic drug molecule-iodinated oil dispersion can be largely held in the liver cancer area for a long time, while the dispersion will be completely metabolized in the normal tissues, thereby realizing a co-treatment of long-term chemotherapy and radiosensitization for the liver cancer area.

In another embodiment, a photosensitive drug molecule is used in the method to prepare a photosensitive drug/chemotherapeutic drug-iodinated oil dispersion or a photosensitive drug/radiotherapeutic drug-iodinated oil dispersion, which leads to an embolization in the hepatic feeding artery. When the dispersion in the normal tissues is completely metabolized, there is still a certain amount of the dispersion remaining in the tumor region, so that the surgical resection and/or phototherapy navigated by molecular imaging can be performed at the tumor region.

The beneficial effects of the invention are described as follows.

1. SHIFT is used to prepare a drug-iodinated oil dispersion herein, which enables the complete mixing of the iodinated oil and the drug molecules and the stable and uniform dispersion of the drug molecules in the iodinated oil. No obvious sedimentation is observed after the drug-iodinated oil dispersion prepared herein is stored for two weeks, and after stored for 12 days, the dispersion can still have a fluorescence intensity ¾ or more of the initial value.

2. In the prepared drug-iodinated oil dispersion, the iodinated oil is not negatively affected in the viscosity, solubility and diffusivity, so that it can be effectively used in the embolization treatment of liver cancer.

3. In the use of the drug-iodinated oil dispersion for embolization treatment, the drug is slowly released to realize the co-treatment, or the surgical resection navigated by molecular imaging can be performed after the liver cancer area is reduced through the initial therapy.

### BRIEF DESCRIPTION OF THE DRAWINGS

FIG. 1A shows absorption curves of iodinated oil (IO), indocyanine green (ICG), doxorubicin (Dox), doxorubicin-iodinated oil dispersion (IO@ICG) and indocyanine green/doxorubicin-iodinated oil dispersion (IO@ICG/Dox).

FIG. 1B shows pictures of IO@ICG and the IO@ICG/Dox after stored at body temperature for two weeks.

FIG. 2 shows fluorescence imaging and intensity of ICG and IO@ICG.

FIG. 3 shows changes of the fluorescence intensity of the ICG and the IO@ICG over time.

### DETAILED DESCRIPTION OF EMBODIMENTS

The application will be more clearly illustrated with reference to the following embodiments.

1) A doxorubicin-iodinated oil dispersion of the application is prepared as follows.

(a) 5-10 mg of doxorubicin is weighed and added to 2 mL of ethanol, then the reaction mixture is mixed uniformly under ultrasonication in an ultrasonic cleaner for 5 min to produce a doxorubicin-ethanol solution.

(b) 2-5 mL of an iodinated oil injection is added to a high-pressure reactor.

(c) 100-200 μL of the doxorubicin-ethanol solution is added to the high-pressure reactor.

(d) The high-pressure reactor is sealed, and then pressurized to 10-20 Mpa and controlled at 30-50° C. by a system. After the pressure and temperature are stable, the reaction mixture is stirred by a stirring paddle at 8,000-12,000 rpm for 0.5-2 h.

(e) The high-pressure reactor is slowly depressurized by discharging air, and the doxorubicin-iodinated oil dispersion is collected.

2) An indocyanine green-iodinated oil dispersion of the application is prepared as follows.

(a) 0.5-1 mg of indocyanine green is weighed and added to 2 mL of ethanol, then the reaction mixture is mixed uniformly under ultrasonication in an ultrasonic cleaner for 5 min to produce an indocyanine green-ethanol solution.

(b) 2-5 mL of the iodinated oil injection is added to a high-pressure reactor.

(c) 100-200 μL of the indocyanine green-ethanol solution is added to the high-pressure reactor.

(d) The high-pressure reactor is sealed, and then pressurized to 10-20 Mpa and controlled at 30-50° C. by the system. After the pressure and temperature are stable, the reaction mixture is stirred by a stirring paddle at 8,000-12,000 rpm for 0.5-2 h.

(e) The high-pressure reactor is slowly depressurized by discharging air, and the indocyanine green-iodinated oil dispersion is collected.

3) An indocyanine green/doxorubicin-iodinated oil dispersion of the application is prepared as follows.

(a) 0.5-1 mg of indocyanine green and 0.5-1 mg of doxorubicin are added to 2 mL of ethanol, and then the reaction mixture is mixed uniformly under ultrasonication in an ultrasonic cleaner for 5 min to produce an indocyanine green/doxorubicin-ethanol solution.

(b) 2-5 mL of an iodinated oil injection is added to a high-pressure reactor.

(c) 100-200 μL of the indocyanine green/doxorubicin-ethanol solution is added to the high-pressure reactor.

(d) The high-pressure reactor is sealed, and then pressurized to 10-20 Mpa and controlled at 30-50° C. by the system. After the pressure and temperature are stable, the reaction mixture is stirred by a stirring paddle at 8,000-12,000 rpm for 0.5-2 h.

(e) The high-pressure reactor is slowly depressurized by discharging air, and the indocyanine green/doxorubicin-iodinated oil dispersion is collected.

In the preparation of above drug-iodinated oil dispersions, no significant variation is observed in the viscosity, solubility and diffusibility of the iodinated oil.

4) A use method of a drug-iodinated oil dispersion in the co-therapy involving embolization for liver cancer includes the following steps.

(a) A doxorubicin-iodinated oil dispersion is injected in the hepatic feeding artery to perform embolization, so that the doxorubicin-iodinated oil dispersion can be held in large quantities in the liver cancer area for a long time to slowly release chemotherapeutic drug molecules, thereby realizing a long-term co-treatment of embolization and chemotherapy.

(b) An indocyanine green-iodinated oil dispersion or an indocyanine green/doxorubicin-iodinated oil dispersion is injected in the hepatic feeding artery to perform embolization. Meanwhile, the tumor area is monitored using magnetic resonance imaging. After the embolization is performed for 2-4 weeks, the tumor area is significantly reduced, and at this time, the surgical resection navigated by holographic molecular imaging can be performed to the tumor area through the indocyanine green fluorescence imaging.

### EXAMPLE 1

A doxorubicin-iodinated oil dispersion was prepared through the following steps.

(a) 5 mg of doxorubicin was weighed and added to 2 mL of ethanol, then the reaction mixture was mixed uniformly under ultrasonication in an ultrasonic cleaner for 5 min to produce a doxorubicin-ethanol solution.

(b) 2 mL of an iodinated oil injection was added to a high-pressure reactor.

(c) 200 μL of the doxorubicin-ethanol solution was added to the high-pressure reactor.

(d) The high-pressure reactor was sealed, and then pressurized to 10 Mpa and controlled at 30° C. by a system. After the pressure and temperature were stable, the reaction mixture was stirred by a stirring paddle at 9,000 rpm for 1 h.

(e) The high-pressure reactor was slowly depressurized by discharging air, and the doxorubicin-iodinated oil dispersion was collected.

### EXAMPLE 2

An indocyanine green-iodinated oil dispersion was prepared through the following steps.

(a) 1 mg of indocyanine green was added to 2 mL of ethanol, then the reaction mixture was mixed uniformly under ultrasonication in an ultrasonic cleaner for 5 min to produce an indocyanine green-ethanol solution.

(b) 5 mL of an iodinated oil injection was added to a high-pressure reactor.

(c) 150 μL of the indocyanine green-ethanol solution was added to the high-pressure reactor.

(d) The high-pressure reactor was sealed, and then pressurized to 20 Mpa and controlled at 40° C. by a system. After the pressure and temperature were stable, the reaction mixture was stirred by a stirring paddle at 8,000 rpm for 2 h.

(e) The high-pressure reactor was slowly depressurized by discharging air, and the indocyanine green-iodinated oil dispersion was collected.

### EXAMPLE 3

An indocyanine green/doxorubicin-iodinated oil dispersion was prepared through the following steps.

(a) 0.75 mg of indocyanine green and 0.75 mg of doxorubicin were added to 2 mL of ethanol, then the reaction mixture was mixed uniformly under ultrasonication in an ultrasonic cleaner for 5 min to produce an indocyanine green/doxorubicin-ethanol solution.

(b) 2.5 mL of an iodinated oil injection was added to a high-pressure reactor.

(c) 150 μL of the indocyanine green/doxorubicin-ethanol solution was added to the high-pressure reactor.

(d) The high-pressure reactor was sealed, then pressurized to 15 Mpa and controlled at 50° C. by a system. After the pressure and temperature were stable, the reaction mixture was stirred by a stirring paddle at 12,000 rpm for 0.5 h.

(e) The high-pressure reactor was slowly depressurized by discharging air, and the indocyanine green/doxorubicin-iodinated oil dispersion was collected.

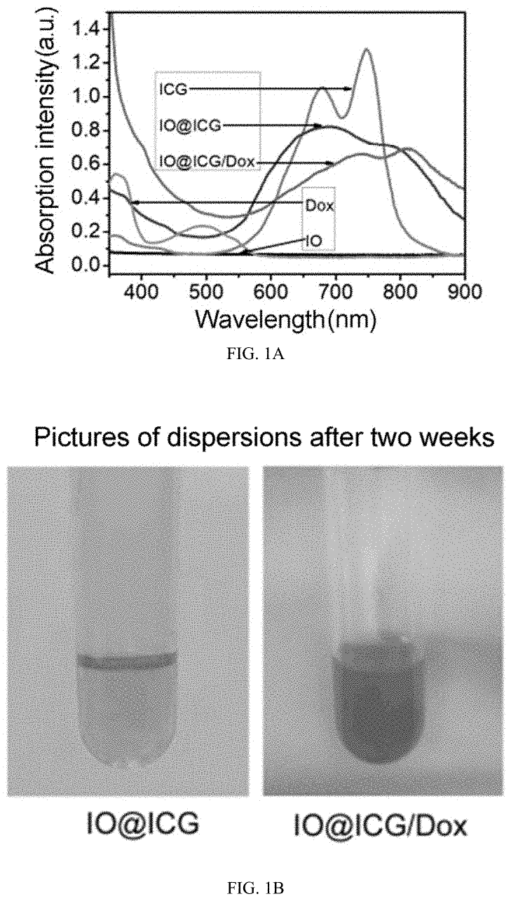

As shown in FIG. 1A, compared to the pure ICG molecule, the ICG molecule in IO@ICG has a different absorption curve, however, an ICG characteristic peak was still observed. Compared to the pure ICG molecule and pure Dox molecule, ICG and Dox molecules in IO@ICG/Dox have different absorption curves, however, ICG and Dox characteristic peaks can still be observed. Moreover, in the IO@ICG and the IO@ICG/Dox, the ICG characteristic peak becomes narrower, which was similar to the ICG characteristic peak shown in the absorption curve of the nanostructure constructed by pure ICG.

As shown in FIG. 1B, the IO@ICG and the IO@ICG/Dox dispersions were respectively allowed to stand for two weeks at ambient temperature, and no significant sedimentation appeared, showing good stability of the IO@ICG and IO@ICG/Dox dispersions.

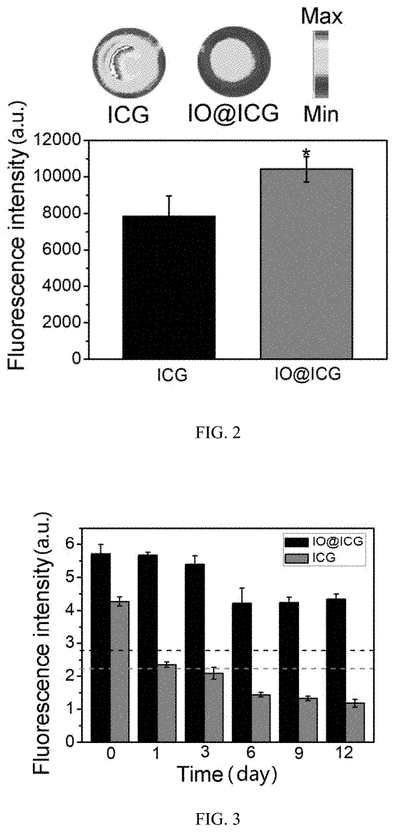

As shown in FIG. 2, IO@ICG was found to have obvious fluorescence emission when excited at 650 nm. Moreover, by comparing the fluorescence intensities of the IO@ICG and ICG under the excitation, IO@ICG had a significantly increased fluorescence intensity at this wavelength than ICG.

As shown in FIG. 3, the ICG had a gradually decreased fluorescence intensity over time, and a fluorescence half-life of the ICG was about three days. Though the prepared IO@ICG also had a gradually decreased fluorescence intensity over time, it can maintain a higher fluorescence intensity for a longer time. After placed for 12 days, the fluorescence intensity of the IO@ICG was still maintained at 3/4 or more of the initial value. These results fully demonstrated that the IO@ICG prepared in the application has excellent stability over time.

In the application, a drug-iodinated oil dispersion is prepared through the SHIFT. Compared to drug-iodinated oil suspensions or emulsions, the drug-iodinated oil dispersion has controllable concentration and dispersibility, better stability and predictable release performance. In addition, the drug will not significantly affect the physical properties of the iodinated oil, enabling the application of the drug-iodinated oil dispersion in the embolization treatment for liver cancer. Therefore, the drug-iodinated oil dispersion of the invention can be applied in the co-therapy of embolization and drug for liver cancer.

What is claimed is:

1. A drug-iodinated oil dispersion, comprising a drug molecule and an iodinated oil injection;
wherein the drug molecule has a concentration of 0.05-5 mg/mL in the drug-iodinated oil dispersion;
the drug-iodinated oil dispersion is prepared through the following steps:
1) dispersing the drug molecule in a first solvent to obtain a solution A;
2) adding the iodinated oil injection and the solution A to a high-pressure reactor;
3) boosting a pressure of the high-pressure reactor to 10-20 Mpa and controlling a temperature of the high-pressure reactor at 30-50° C.; and stirring the reaction mixture for 0.5-2 h after the pressure and the temperature are stable; and
4) depressurizing the high-pressure reactor; and collecting the drug-iodinated oil dispersion from the high-pressure reactor.

2. The drug-iodinated oil dispersion of claim 1, wherein the iodinated oil injection comprises 37.0%-41.0% by weight of iodine.

3. The drug-iodinated oil dispersion of claim 1, wherein the first solvent is ethanol.

4. The drug-iodinated oil dispersion of claim 3, wherein the drug molecule has a concentration of 0.5-10 mg/mL in the solution A, and a volume ratio of the solution A to the iodinated oil injection is 0.01-0.5:1-10.

5. The drug-iodinated oil dispersion of claim 1, wherein the drug molecule is selected from a chemotherapeutic drug molecule, a radiotherapeutic drug molecule, a radiotherapy-sensitized chemotherapeutic drug molecule, a photosensitive drug molecule, or a combination thereof.

6. The drug-iodinated oil dispersion of claim 5, wherein the chemotherapeutic drug molecule comprises at least one of epirubicin, 5-fluorouridine, pirarubicin, pingyangmycin, gemcitabine, camptothecin and paclitaxel.

7. The drug-iodinated oil dispersion of claim 5, wherein the radiotherapeutic drug molecule comprises at least one of ¹³¹I-, ¹⁷⁷Lu- and ⁶⁴Cu-labeled radiotherapeutic molecules.

8. The drug-iodinated oil dispersion of claim 5, wherein the radiotherapy-sensitized chemotherapeutic drug molecule comprises at least one of docetaxel, curcumin, tirapazamine, cisplatin, doxorubicin and mitomycin C.

9. The drug-iodinated oil dispersion of claim 5, wherein the photosensitive drug molecule comprises at least one of indocyanine green, new indocyanine green IR-820, 5-aminolevulinic acid and porphyrin dye.

10. A use method of the drug-iodinated oil dispersion of claim 1, comprising:
applying the drug-iodinated oil dispersion in the preparation of a tumor-treating drug or an embolic agent.

11. A method for preparing a drug-iodinated oil dispersion, comprising:
1) dispersing a drug molecule in ethanol to obtain a solution A;
2) adding an iodinated oil injection and the solution A to a high-pressure reactor;
3) boosting a pressure of the high-pressure reactor to 10-20 Mpa and controlling a temperature of the high-pressure reactor at 30-50° C.; and stirring the reaction mixture for 0.5-2 h after the pressure and the temperature are stable; and
4) depressurizing the high-pressure reactor; and collecting the drug-iodinated oil dispersion from the high-pressure reactor.
